(19) United States
(12) Patent Application Publication
(10) Pub. No.: US 2025/0257152 A1
(43) Pub. Date: Aug. 14, 2025

(54) METHOD FOR PREPARING NANOCELLULOSE USING NONIONIC SURFACTANTS

(71) Applicants: HYUNDAI MOTOR COMPANY, Seoul (KR); KIA CORPORATION, Seoul (KR); INSTITUTE FOR RESEARCH & INDUSTRY COOPERATION PUSAN NATIONAL UNIVERSITY, Busan (KR)

(72) Inventors: Sung Wan Jeon, Suwon-si (KR); Jin Woo Choung, Suwon-si (KR); Jong Geun Sung, Hwaseong-si (KR); Il Kim, Busan (KR); Jae Eon Jeong, Daejeon (KR)

(73) Assignees: HYUNDAI MOTOR COMPANY, Seoul (KR); KIA CORPORATION, Seoul (KR); INSTITUTE FOR RESEARCH & INDUSTRY COOPERATION PUSAN NATIONAL UNIVERSITY, Busan (KR)

(21) Appl. No.: 19/171,037

(22) Filed: Apr. 4, 2025

Related U.S. Application Data
(62) Division of application No. 17/523,678, filed on Nov. 10, 2021.

(30) Foreign Application Priority Data
Jun. 15, 2021 (KR) 10-2021-0077148

(51) Int. Cl. C08B 11/02; C07C 69/533; C08L 75/04

(52) U.S. Cl. CPC C08B 11/02; C07C 69/533; C08L 75/04

(57) ABSTRACT

A method of preparing surface-modified nanocellulose includes mixing a nanocellulose aqueous solution with a nanocellulose surface modifier, centrifuging a mixed solution, and removing an amount of water after centrifugation, where the nanocellulose surface modifier is a nonionic surfactant in which a hydrophilic group is subjected to ring opening with glycidol.

### CROSS-REFERENCE TO RELATED APPLICATION

This application is a divisional of U.S. application Ser. No. 17/523,678 filed on Nov. 10, 2021, which claims under 35 U.S.C. § 119(a) the benefit of priority from Korean Patent Application No. 10-2021-0077148, filed on Jun. 15, 2021, the disclosures of which are incorporated herein by reference in their entirety.

### BACKGROUND

### (a) Technical Field

The present disclosure relates to a technique for physical interaction of nanocellulose, and more particularly to a method of separating nanocellulose from water in an aqueous solution using a physical interaction promoter, and to the synthesis of a cellulose interaction promoter capable of increasing the dispersibility of nanocellulose in a polymer matrix and improving the physical properties of a polymer composite material.

### (b) Background Art

With the recent success in the economical manufacture of certain nano-sized fibers from wood in various ways, cellulose, the most abundant polymer material on the planet, is receiving attention as a new reinforcing agent that is able to replace carbon fiber or glass fiber for use as a reinforcing agent for a general polymer composite material.

Typically, fibrous nanocellulose (NC) has a large surface area and a high aspect ratio. Thus, NC has excellent mechanical properties, as well as eco-friendliness, biodegradability, and biosynthesis capability. As a result, it is useful as an eco-friendly and renewable material when used as a reinforcing agent for composite materials.

Currently, automobile-related companies around the world are constantly striving to reduce the weight of automobiles. Polyurethane, which is used as a representative industrial material for automobiles, is mainly made from petroleum-derived polyol and isocyanate, such as toluene diisocyanate (TDI) or methylene diphenyl diisocyanate (MDI). However, the use of existing petroleum polymer materials is restricted by the introduction of a carbon tax due to the entry into force of the Kyoto Protocol in 2013. Automakers have established strategies for using sustainable plastics for various polymer materials used in the manufacture of automobiles. The global automotive industry is actively responding to increasingly stringent environmental regulations. Recently, a green carbon-based polyol using vegetable oil as a raw material has been devised and is produced mainly in Germany. Ford Motor Company now manufactures 5% of Mustang car seats with soybean-oil-based foam using soybean-oil-based BioPolyol, having high natural oil polyol (NOP) content. For one small car, 150 kg of plastic is usually used and polyurethane is used in an amount of 15 to 20% of the total plastic. The average amount of polyurethane used for automobiles is 22.5 to 30 kg. According to Utech data, the use of BioPolyol in lieu of petroleum-derived polyurethane is expected to prevent the emission of 272.2 tons of carbon dioxide per year. When the amount of BioPolyol-derived polyurethane is increased, the physical properties of polyurethane are decreased compared to pure polyurethane. In order to compensate therefor, microcrystalline cellulose is added to or charged in polyurethane.

However, cellulose has strong hydrophilic properties due to the hydroxyl group (—OH) on the surface thereof, is difficult to separate from water in an aqueous solution and is not evenly dispersed but agglomerates or precipitates quickly when mixed with a hydrophobic polymer matrix.

With the goal of solving such problems, a conventional method for surface modification of nanocellulose imparts hydrophobicity through chemical modification of the hydroxyl group on the surface thereof or through modification by electrostatic attraction using a cationic surfactant.

The chemical modification method typically uses a method of modifying a hydroxyl group using an isocyanate functional group and a urethane bond. In this case, a large amount of energy is required for heating or freezing in the process of completely removing water from the nanocellulose dispersed in water. Moreover, taking into consideration all of organic solvents, catalysts, and reactants for chemical modification, this method is unsuitable for large-scale surface modification of cellulose due to the complexity of the synthesis process and economic problems. Alternatively, chemical modification methods known to impart hydrophobicity, including a method using alkenyl succinic anhydride, are also unsuitable for large-scale surface modification of nanocellulose due to the aforementioned problems. Accordingly, surface modification using the electrostatic attraction of a cationic surfactant such as cetyltrimethylammonium bromide (CTAB) or dimethyldioctadecylammonium chloride (DODMAC) has been reported. However, there is difficulty in solving problems due to side reactions that may be caused by the cation used as a surface modifier in subsequent synthesis processes.

As interest in the environment is gradually increasing around the world, efforts to synthesize eco-friendly, renewable, and sustainable materials in lieu of petroleum-based polymers are continuing. Based on interest therein in the global market, the present inventors have made great efforts to produce, as a reinforcing agent for polymer composite materials, nanocellulose having properties such as eco-friendly properties, improved mechanical strength of composite materials, lighter weight, etc. by obtaining raw materials from plants. The preset inventors have also made great efforts to develop a processing system therefor, thus culminating in the present disclosure.

Accordingly, the present disclosure has been made keeping in mind the problems encountered in the related art. The present disclosure is intended to provide novel nanocellulose modified with a surface modifier based on a nonionic surfactant and to provide a method of preparing the same.

An aspect of the present disclosure is to provide a novel nonionic surfactant for modifying nanocellulose and to provide a method of preparing the same.

Another aspect of the present disclosure is to provide a method of preparing nanocellulose modified with the novel surface modifier and nanocellulose prepared using the method.

Still another aspect of the present disclosure is to provide a polymer matrix having improved dispersibility of nanocellulose and to provide a method of preparing the same.

Specifically, the disclosure is intended to provide a method of preparing an interaction promoter capable of precipitation and dispersion of a large amount of cellulose. The method makes it possible to separate water and nanocellulose in an aqueous solution using a physical interaction promoter for cellulose. The method also makes it possible to increase the dispersibility of cellulose in a polymer matrix while also improving the physical properties of a polymer composite material, and a method of precipitating and dispersing cellulose.

In order to solve the above problems, the following solutions of the present disclosure are provided.

In one aspect, the present disclosure provides a nanocellulose surface modifier, in which the nanocellulose surface modifier is a nonionic surfactant in which a hydrophilic group is subjected to ring opening with glycidol.

In an embodiment, the nonionic surfactant is prepared through a ring-opening reaction of at least one compound selected from among a carboxylic acid compound having 4 to 25 carbon atoms, an alcohol compound having 4 to 25 carbon atoms, and an amine compound having 4 to 25 carbon atoms, with glycidol.

In another embodiment, the carboxylic acid compound having 4 to 25 carbon atoms is at least one compound selected from among butyric acid, valeric acid, caproic acid, enatic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, alpha-linolenic acid, stearidonic acid, eicosapentaenoic acid, docosahexaenoic acid, linoleic acid, gamma-linoleic acid, dihomo-gamma-linolenic acid, arachidonic acid, docosatetraenoic acid, palmitoleic acid, vaccenic acid, paulinic acid, oleic acid, elaidic acid, gondoic acid, erucic acid, nervonic acid, and mead acid. The alcohol compound having 4 to 25 carbon atoms is at least one compound selected from among butanol, pentanol, hexanol, heptanol, octanol, nonanol, decanol, undecanol, dodecanol, tridecanol, tetradecanol, pentadecanol, palmityl alcohol, octadecanol, nonadecanol, eicosanol, heneicosanol, docosanol, tricosanol, tetracosanol, and pentacosanol. The amine compound having 4 to 25 carbon atoms is at least one compound selected from among butanamine, pentanamine, hexanamine, heptanamine, octanamine, nonanamine, decanamine, undecanamine, dodecanamine, tridecanamine, tetradecanamine, pentadecanamine, hexadecanamine, heptadecanamine, octadecanamine, nonadecanamine, eicosanamine, heneicosanamine, docosanamine, tricosanamine, tetracosanamine, and pentacosanamine.

In still another embodiment, the nanocellulose surface modifier is in a hyperbranched polyglycidol (HBPG) form.

In yet another embodiment, the nanocellulose surface modifier is prepared by reacting at least one compound selected from among a carboxylic acid compound having 4 to 25 carbon atoms, an alcohol compound having 4 to 25 carbon atoms, and an amine compound having 4 to 25 carbon atoms with glycidol at a molar ratio of 1:1-30.

In still yet another embodiment, the nanocellulose surface modifier is prepared by reacting a carboxylic acid compound having 4 to 25 carbon atoms and glycidol at a molar ratio of 1:1-8.

In a further embodiment, the nanocellulose surface modifier has 1 to 10 hydroxyl groups.

The present disclosure provides a method of preparing the nanocellulose surface modifier of any one of the above embodiments according to one aspect of the present disclosure including adding at least one compound selected from among a carboxylic acid compound having 4 to 25 carbon atoms, an alcohol compound having 4 to 25 carbon atoms, and an amine compound having 4 to 25 carbon atoms and a catalyst, performing nitrogen purging at 80° C. to 120° C. for 30 minutes to 5 hours, and adding glycidol thereto and then carrying out a reaction for 4 to 48 hours.

In another aspect, the present disclosure provides a method of preparing surface-modified nanocellulose including mixing a nanocellulose aqueous solution with the nanocellulose surface modifier described above, centrifuging the mixed solution, and removing a large amount of water after centrifugation.

In an embodiment, the nanocellulose included in the nanocellulose aqueous solution and the nanocellulose surface modifier are mixed at a weight ratio of 1:0.2-3.

In still another aspect, the present disclosure provides surface-modified nanocellulose prepared using the method described above.

In an embodiment, the surface-modified nanocellulose is in a slurry form.

In another embodiment, the surface-modified nanocellulose is configured such that the surface modifier and the nanocellulose are joined through hydrogen bonding.

In yet another aspect, the present disclosure provides a method of preparing a polyol polymer matrix having nanocellulose dispersed therein, including dispersing, injecting or adding the nanocellulose surface-modified with the nanocellulose surface modifier described above to a polyol.

In an embodiment, the method further includes removing water from the mixed solution.

In another embodiment, the surface-modified nanocellulose is added in an amount of 0.1 wt % to 10 wt % to the polyol based on the total weight of the polyol polymer matrix.

In still yet another aspect, the present disclosure provides a polyol polymer matrix having nanocellulose dispersed therein, prepared using the method described above.

In an embodiment, the polyol polymer matrix has increased tensile strength and heat resistance.

### BRIEF DESCRIPTION OF THE DRAWINGS

The above and other features of the present disclosure are now described in detail with reference to certain embodiments thereof illustrated in the accompanying drawings, which are given hereinbelow by way of illustration only, and thus are not limitative of the present disclosure, and wherein:

FIG. 1 schematically shows the surface modification of cellulose according to the present disclosure, in which nanocellulose is present in a form dispersed in water during the preparation process (it is difficult to fibrillate cellulose to a nano diameter in a dry method). In order to separate nanocellulose in the state of being dispersed in water from water, an interaction promoter (e.g. a surface modifier) is prepared and added thereto, the nanocellulose and the surface modifier are precipitated through physical hydrogen bonding, and the surface of the nanocellulose+the interaction promoter is hydrophobic due to the alkyl group of the interaction promoter. The material thus obtained is dispersed in a polymer (e.g. polyol), etc., thereby increasing the dispersibility due to appropriate mutual polarity;

FIG. 2 depicts photographs showing separation of nanocellulose and water;

FIG. 3 shows the results of Test Example 1;

FIG. 4 shows the results of Test Example 2-1;

FIG. 5 shows the results of Test Example 2-2;

FIG. 6 shows the results of Test Example 3;

FIG. 7 shows the results of Test Example 4;

FIG. 8 shows the results of Test Example 5;

FIG. 9 shows the results of Test Example 6;

FIG. 10 shows the results of Test Example 7;

FIGS. 11A and 11B show the results of Test Example 8; and

FIG. 12 shows the results of Test Example 9.

### DETAILED DESCRIPTION

Where the gist of the present disclosure may have been obscured, a description of well-known configurations and functions has been omitted. In the present specification, the terms “comprising” or “including” mean that other components may be further included unless otherwise specified.

In the present specification, when a range is described for a variable, it should be understood that the variable includes all values including the end points described within the stated range. For example, the range of “5 to 10” should be understood to include any subranges, such as 6 to 10, 7 to 10, 6 to 9, 7 to 9, and the like, as well as individual values of 5, 6, 7, 8, 9 and 10, and should also be understood to include any value between valid integers within the stated range, such as 5.5, 6.5, 7.5, 5.5 to 8.5, 6.5 to 9 and the like. Also, for example, the range of “10% to 30%” should be understood to include subranges, such as 10% to 15%, 12% to 18%, 20% to 30%, etc., as well as all integers including values of 10%, 11%, 12%, 13% and the like up to 30%, and should also be understood to include any value between valid integers within the stated range, such as 10.5%, 15.5%, 25.5%, and the like.

Hereinafter, a detailed description is given of the present disclosure.

In the present disclosure, surface modification serves to change the functional group present on the surface of nanocellulose. In one example, a hydrophilic functional group is changed to a non-hydrophilic or hydrophobic functional group, or an amine functional group is added thereto, but the present disclosure is not limited thereto. Moreover, in another example, surface modification is carried out using a nonionic surfactant that is subjected to ring opening with glycidol described in the present disclosure, but the present disclosure is not limited thereto.

A method of preparing a physical interaction promoter for cellulose using a surfactant according to the present disclosure includes preparing an interaction promoter, mixing a dispersed nanocellulose solution with the interaction promoter to separate the same from water, and centrifuging the stirred mixed solution, followed by dispersing the surface-modified nanocellulose in a polymer matrix.

The present disclosure provides a nanocellulose surface modifier, a method of precipitating (e.g. separating or preparing) nanocellulose using the same, and a method of dispersing the surface-modified nanocellulose in a polymer, as described below.

[Method of Preparing Interaction Promoter](HBPG: Hyperbranched Polyglycidol, in which Glycidol is Bound to a Functional Group Having an Alkyl Chain)

In one embodiment, the present disclosure provides a method of preparing a nanocellulose surface modifier that is an interaction promoter. The method includes: (a) adding, as a starting material, any one material selected from among organic materials capable of substituting a functional group with a hydroxyl group through a ring-opening reaction of glycidol in a functional group having an alkyl chain and a catalyst; (b) adding glycidol dropwise after purging with nitrogen at 100° C. for 1 hour; and (c) carrying out a reaction for 24 hours to afford an interaction promoter.

### [Method of Precipitating Nanocellulose Using Interaction Promoter]

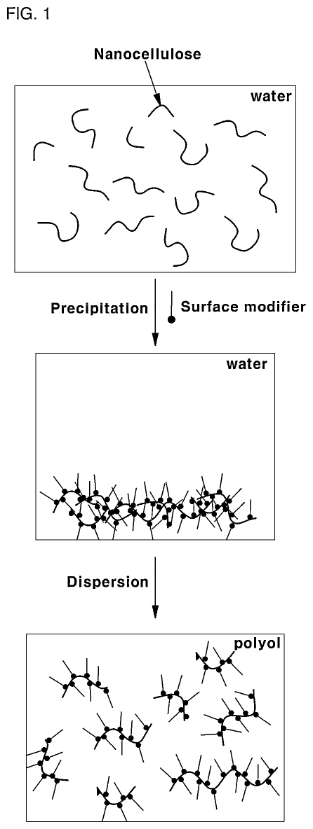

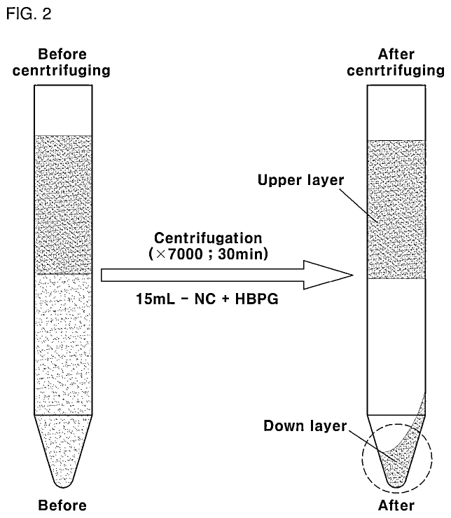

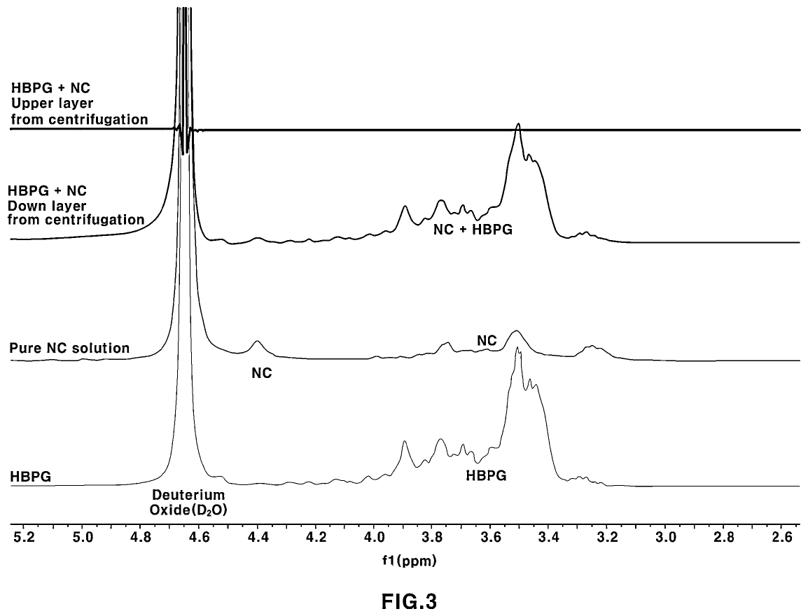

In one embodiment, the present disclosure provides a method of precipitating nanocellulose. The method includes (a) mixing a nanocellulose aqueous solution and the interaction promoter (FIG. 1), (b) centrifuging the mixed solution (FIGS. 2 and 3), and (c) removing a large amount of water after centrifugation to afford surface-modified nanocellulose in a slurry state including a small amount of water.

### [Method of Dispersing Nanocellulose in Polymer]

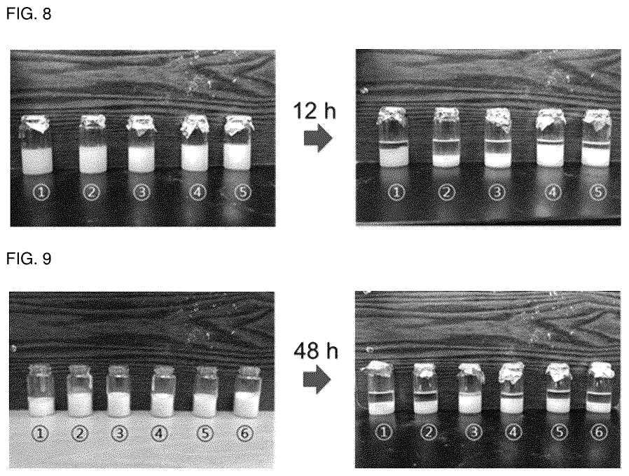

In one embodiment, the present disclosure provides a method of preparing a polymer matrix having nanocellulose dispersed therein. The method includes (a) mixing the nanocellulose in a slurry form and a polyol and (b) preparing a polyol in which cellulose is dispersed by completely removing water (FIG. 8).

Therefore, the present disclosure is intended to enable separation of water and nanocellulose in an aqueous solution by applying a surface modifier having a physical interaction promotion effect to typical nanocellulose. In addition, the present disclosure is intended to provide a method of increasing the dispersibility of the separated nanocellulose in the polymer matrix while also improving the physical properties of the polymer composite material. In addition, the present disclosure is intended to provide a method of preparing the nanocellulose surface modifier (e.g. interaction promoter) capable of precipitation and dispersion of a large amount of cellulose and a method of precipitating and dispersing cellulose.

According to the present disclosure, a nonionic surfactant is added to the nanocellulose aqueous solution, thus introducing an alkyl chain exhibiting the hydrophobicity of the cellulose as well as forming a physical hydrogen bond with a functional group on the surface of the cellulose (FIG. 1). Thereby, it is possible to increase the dispersibility in the polymer matrix by increasing the precipitation in the aqueous solution and the hydrophobicity of the cellulose. Moreover, it is possible to control the ratio of hydrophilicity and hydrophobicity by adjusting the number of hydroxyl groups of the surfactant so as to be suitable for the characteristics of the polymer matrix, increase dispersibility in the polymer matrix. It is also possible to improve the physical properties of the polymer composite material (FIGS. 9, 10, 11, 12, and 13).

The physical interaction according to the present disclosure is a method of modifying the surface of nanocellulose using only intermolecular physical bonding, thereby minimizing side reactions in subsequent processes.

In addition, the process according to the present disclosure is advantageous in that the surface modification process may be performed without a chemical reaction at a relatively low temperature using a surface modifier that is synthesized from an inexpensive starting material. Thereby, it is possible to conduct a surface modification process for a large amount of nanocellulose.

In a variety of commercially available nonionic surfactants having functional groups such as hydroxyl, carboxyl, and amine groups, the hydroxyl group is changed only through glycidol ring-opening polymerization, and the extent of hydrophilicity or hydrophobicity of the nonionic surfactant may be changed. Thereby, it is possible to appropriately disperse the cellulose in various polymers.

Hereinafter, various aspects of the present disclosure are described.

In one aspect, the present disclosure provides a nanocellulose surface modifier, in which the nanocellulose surface modifier is a nonionic surfactant in which a hydrophilic group is subjected to ring opening with glycidol.

In an embodiment, the nonionic surfactant is prepared through a ring-opening reaction of at least one selected from among a carboxylic acid compound having 4 to carbon atoms, an alcohol compound having 4 to 25 carbon atoms, and an amine compound having 4 to 25 carbon atoms with glycidol.

In another embodiment, the carboxylic acid compound having 4 to 25 carbon atoms is at least one compound selected from among butyric acid, valeric acid, caproic acid, enatic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, alpha-linolenic acid, stearidonic acid, eicosapentaenoic acid, docosahexaenoic acid, linoleic acid, gamma-linoleic acid, dihomo-gamma-linolenic acid, arachidonic acid, docosatetraenoic acid, palmitoleic acid, vaccenic acid, paulinic acid, oleic acid, elaidic acid, gondoic acid, erucic acid, nervonic acid, and mead acid. The alcohol compound having 4 to 25 carbon atoms is at least one compound selected from among butanol, pentanol, hexanol, heptanol, octanol, nonanol, decanol, undecanol, dodecanol, tridecanol, tetradecanol, pentadecanol, palmityl alcohol, octadecanol, nonadecanol, eicosanol, heneicosanol, docosanol, tricosanol, tetracosanol, and pentacosanol. The amine compound having 4 to 25 carbon atoms is at least one compound selected from among butanamine, pentanamine, hexanamine, heptanamine, octanamine, nonanamine, decanamine, undecanamine, dodecanamine, tridecanamine, tetradecanamine, pentadecanamine, hexadecanamine, heptadecanamine, octadecanamine, nonadecanamine, eicosanamine, heneicosanamine, docosanamine, tricosanamine, tetracosanamine, and pentacosanamine.

In still another embodiment, the nanocellulose surface modifier is in a hyperbranched polyglycidol (HBPG) form.

In yet another embodiment, the nanocellulose surface modifier is prepared by reacting at least one compound selected from among a carboxylic acid compound having 4 to 25 carbon atoms, an alcohol compound having 4 to 25 carbon atoms, and an amine compound having 4 to 25 carbon atoms with glycidol at a molar ratio of 1:1-30.

In still yet another embodiment, the nanocellulose surface modifier is prepared by reacting a carboxylic acid compound having 4 to 25 carbon atoms and glycidol at a molar ratio of 1:1-8.

In a further embodiment, the nanocellulose surface modifier has 1 to 10 hydroxyl groups.

The present disclosure provides a method of preparing the nanocellulose surface modifier of any one of the above embodiments according to one aspect of the present disclosure. The method includes: adding at least one compound selected from among a carboxylic acid compound having 4 to 25 carbon atoms, an alcohol compound having 4 to 25 carbon atoms, and an amine compound having 4 to 25 carbon atoms and a catalyst; performing nitrogen purging at 80° C. to 120° C. for 30 minutes to 5 hours; and adding glycidol thereto and then carrying out a reaction for 4 to 48 hours.

In another aspect, the present disclosure provides a method of preparing surface-modified nanocellulose. The method includes mixing a nanocellulose aqueous solution with the nanocellulose surface modifier described above, centrifuging the mixed solution, and removing a large amount of water after centrifugation.

In an embodiment, the nanocellulose included in the nanocellulose aqueous solution and the nanocellulose surface modifier are mixed at a weight ratio of 1:0.2-3.

In still another aspect, the present disclosure provides surface-modified nanocellulose prepared using the method described above.

In an embodiment, the surface-modified nanocellulose is in a slurry form.

In another embodiment, the surface-modified nanocellulose is configured such that the surface modifier and the nanocellulose are joined through hydrogen bonding.

In yet another aspect, the present disclosure provides a method of preparing a polyol polymer matrix having nanocellulose dispersed therein. The method includes dispersing, injecting, or adding the nanocellulose surface-modified with the nanocellulose surface modifier described above to a polyol.

In an embodiment, the method further includes removing water from the mixed solution.

In another embodiment, the surface-modified nanocellulose is added in an amount of 0.1 wt % to 10 wt % to the polyol based on the total weight of the polyol polymer matrix.

In still yet another aspect, the present disclosure provides a polyol polymer matrix having nanocellulose dispersed therein, prepared using the method described above.

In an embodiment, the polyol polymer matrix has increased tensile strength and heat resistance.

In another embodiment, the polyol polymer matrix is polyurethane.

A better understanding of the present disclosure may be obtained through the following preparation examples, examples, and test examples. However, these preparation examples, examples, and test examples are merely set forth to illustrate the present disclosure and are not to be construed as limiting the scope of the present disclosure.

### Preparation Example 1: Preparation of Nanocellulose Surface Modifier

### Example 1: Use of Initiator Having Hydroxyl Group Terminal (e.g. Span 80) (Span 80:Glycidol=1:3 Mol Ratio)

### (1) Preparation Method

\textcircled{1} Span 80 (initiator), which is sorbitan monooleate, glycidol (monomer), and double metal cyanide (DMC) (catalyst) were provided.
\textcircled{2} 20 g of Span 80 and 0.03 g of a DMC catalyst (0.3 wt % of glycidol) were placed in a 250 ml round-bottom flask.
\textcircled{3} Water was removed while performing stirring at 90° C. for 1 hour with nitrogen purging (in consideration of volume expansion, and glycidol stored in a refrigerator was allowed to stand at room temperature in advance).
\textcircled{4} After completion of nitrogen purging for 1 hour, 10.37 g of glycidol was pumped into the round-bottom flask at a rate of 1 ml/h using a syringe pump.
\textcircled{5} The reaction was carried out for an additional 2 hours after the addition of glycidol, and whether the reaction was terminated was determined through Proton Nuclear Magnetic Resonance (1H NMR).
\textcircled{6} After completion of the reaction, chloroform was placed in the round-bottom flask to dissolve the product.
\textcircled{7} Polyglycidol and the DMC catalyst, which were byproducts floating in chloroform without being dissolved, were filtered using a filter paper.
\textcircled{8} The chloroform solvent was completely evaporated, followed by sufficient drying in a vacuum oven.

### (2) Preparation Result

Upon ¹H NMR, it was confirmed that the desired product was obtained based on the disappearance of the peak corresponding to glycidol and the increase of the peak corresponding to the hydrophilic group (3.4 to 4.6 ppm).

### Example 2: Use of Initiator Having Carboxyl Group Terminal (Stearic Acid)

### (1) Preparation Method

### Step 1

\textcircled{1} Stearic acid (initiator), glycidol (monomer), and triethylamine (catalyst) were provided.
\textcircled{2} 76.81 g (0.27 mol) of stearic acid (S.A) was placed in a 250 ml round-bottom flask and dissolved at 70° C. (Tm: 69.3° C.).
\textcircled{3} After complete dissolution of S.A, 1.1 g of triethylamine was added thereto, and water was removed while performing stirring at 70° C. for 1 hour with nitrogen purging (in consideration of volume expansion, glycidol stored in a refrigerator was allowed to stand at room temperature in advance).
\textcircled{4} After completion of nitrogen purging for 1 hour, 40 g (0.54 mol) of glycidol was pumped into the round-bottom flask at a rate of 2 ml/min using a syringe pump.
\textcircled{5} The reaction was carried out for a total of 6 hours.
\textcircled{6} After completion of the reaction, chloroform was placed in the round-bottom flask to dissolve the product.
\textcircled{7} Polyglycidol and the DMC catalyst, which were byproducts floating in chloroform without being dissolved, were filtered using a filter paper.
\textcircled{8} The chloroform solvent was placed in a separatory funnel and forcefully mixed with distilled water, followed by purification through layer separation.
\textcircled{9} The chloroform solvent was completely evaporated, followed by sufficient drying in a vacuum oven.

### Step 2

\textcircled{1} Stearoyl glycerol (initiator), glycidol (monomer), and DMC (catalyst) were provided.
\textcircled{2} 50 g of stearoyl glycerol and 0.055 g of a DMC catalyst (0.3 wt/o of glycidol) were placed in a 250 ml round-bottom flask.
\textcircled{3} Water was removed while performing stirring at 100° C. for 1 hour with nitrogen purging (in consideration of volume expansion, glycidol stored in a refrigerator was allowed to stand at room temperature in advance).
\textcircled{4} After completion of nitrogen purging for 1 hour, 18.6053 ml of glycidol was pumped into the round-bottom flask at a rate of 1.0336 ml/h using a syringe pump.
\textcircled{5} The reaction was carried out for an additional 2 hours after the addition of glycidol, and whether the reaction was terminated was determined through ¹H NMR.
\textcircled{6} After completion of the reaction, chloroform was placed in the round-bottom flask to dissolve the product.
\textcircled{7} Polyglycidol and the DMC catalyst, which were byproducts floating in chloroform without being dissolved, were filtered using a filter paper.
\textcircled{8} The chloroform solvent was completely evaporated, followed by sufficient drying in a vacuum oven.

### (2) Preparation Result

Upon ¹H NMR, it was confirmed that the desired product was obtained based on the disappearance of the peak corresponding to glycidol and the increase of the peak corresponding to the hydrophilic group (3.4 to 4.6 ppm).

### Preparation Example 2: Preparation of Surface-Modified Nanocellulose Using Surface Modifier

### (1) Preparation Method

\textcircled{1} 50 wt % of a surface modifier (surfactant) based on the amount of surface-modified nanocellulose fiber (CNF) was added to a 1 wt % CNF suspension, and was heated and sufficiently dispersed (dispersion through heating to 70° C. with stirring).
\textcircled{2} Using a centrifuge (5 min/7000 rpm), the layers were separated, the aqueous supernatant was removed, and a CNF slurry was isolated and obtained.
\textcircled{3} The slurry was added to polyol (poly(tetramethylene ether)glycol (PTMG) or polypropylene glycol (PPG)) and sufficiently dispersed through stirring with a magnetic bar and heating to a temperature of 90° C.

### (2) Preparation Result

The layers were separated using a centrifuge, and water in the upper layer and the CNF slurry in the bottom portion were confirmed.
It was confirmed that the slurry was dispersed stably and for a longer time than polyol (PTMG or PPG) dispersed without the use of a surface modifier.

### Preparation Example 3: Preparation of Polymer Having Surface-Modified Nanocellulose Dispersed Therein

### (1) Preparation Method

### Preparation of CNF/Polyurethane Composite Material

\textcircled{1} 10 g of PTMG1000 in which CNF with a surface modifier was dispersed was sufficiently dewatered (In a closed system, this process was performed at 90° C. for 3 hours with mechanical stirring in a vacuum).
\textcircled{2} The temperature was lowered to 60° C., 3.364 g of hexamethylene diisocyanate (HDI) was added thereto in a nitrogen atmosphere, and a reaction was carried out for 3 hours (NCO/OH=2).
\textcircled{3} The temperature was raised to 100° C., 0.9 g of 1,4-butanediol (BDO) as a chain extender was added thereto, 5 ml of purified dimethylformamide (DMF) and 0.05 ml of a dibutyltin dilaurate catalyst were added thereto, and polymerization was carried out for 1 hour.
\textcircled{4} After polymerization, the polymerized polyurethane was added to DMF and thus made into a liquid form, and was then poured into a mold such as a Teflon beaker or a Petri dish, and DMF was removed at 70° C.

### (2) Preparation Result

Through Fourier transform infrared (FT-IR) measurement at each step, the —NCO peak at 2270 cm⁻¹ was observed, and complete disappearance thereof was confirmed in the final form, based on which the degree of polymerization was determined.
Structural properties were measured through FT-IR, Raman spectroscopy, and x-ray diffraction (XRD).
Mechanical properties were measured through a universal testing machine (UTM).
Thermal properties were measured through differential scanning calorimetry (DSC) and thermogravimetric analysis (TGA).

### Test Example 1. Nuclear Magnetic Resonance Spectroscopy (NMR) Data of Upper and Lower Layers after Centrifugation of Nanocellulose

(1) NMR of the nanocellulose prepared in Preparation Example 2 was measured.
(2) The test results were as shown in FIG. 3. Each of the nanocellulose (NC), the interaction promoter (HBPG), the precipitation portion, and the upper portion was subjected to NMR measurement, indicating that NC and HBPG were present in the precipitation portion and that only water remained in the upper portion.

### Test Example 2-1. Nuclear Magnetic Resonance Spectroscopy (NMR) for Hydroxyl Group Control in Surface Modifier

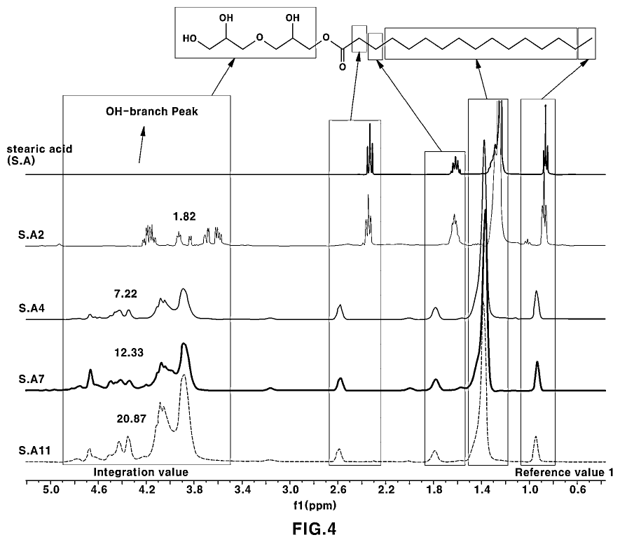

(1) 0.01 g to 0.015 g of a sample of the surface modifier prepared in Preparation Example 1 was sufficiently dissolved in 0.6 ml of CDCl₃ as an NMR solvent and then sampled, followed by ¹H NMR measurement.
(2) The test results were as shown in FIG. 4.
NMR measurement was performed to confirm that the hydrophilic group portion increased depending on the molar ratio of the initiator and glycidol. The peak of the hydrophilic group (polyether backbone+hydroxyl group, 3.4-4.6 ppm) was increased with an increase in the amount of glycidol relative to the terminal methyl group (—CH₃) of the initiator.

### Test Example 2-2. Contact Angle Data of Nanocellulose Film Depending on Hydroxyl Group Control in Surface Modifier

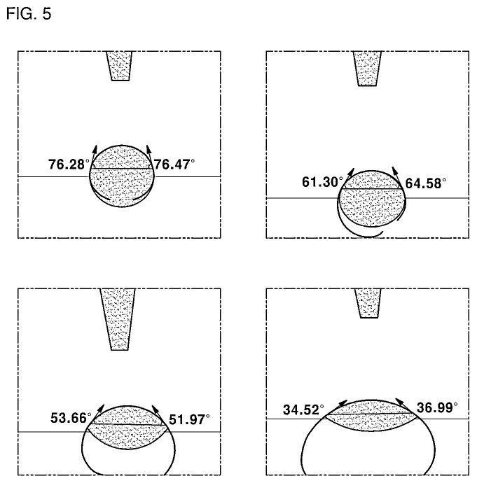

(1) In Test Example 2-1 and FIG. 4, a surface modifier having a controlled hydroxyl group was added to a nanocellulose aqueous solution and precipitated. Then the resulting material was manufactured into a film, the contact angle data of which was then measured.
(2) This test was intended to investigate the increase in the hydrophobicity of the nanocellulose film due to the surface modifier.
(3) In the test method, the contact angle was measured in order to determine the hydrophobic properties of the nanocellulose film due to a change in the surface modifier depending on the molar ratio of the initiator and glycidol.
(4) The test results were as shown in FIG. 5. As seen from the left in FIG. 5, respective nanocellulose films were manufactured after the addition of interaction promoters having 2, 4, 7, and 11 hydroxyl groups (S.A 2, S.A 4, S.A 7, and S.A 11) attached to the alkyl group. NC having various polarities could be obtained by exhibiting hydrophilicity with an increase in the number of glycidols (number of OH groups) toward the right and exhibiting hydrophobicity toward the left.
Because the cellulose film is able to absorb water over time, it was judged that the accuracy was lowered with time. Based on the results of measurement at the same time period, there was a change with or without the addition of the surface modifier, but no tendency depending on changes in the type or amount of the hydrophilic group was observed.

### Test Example 3. Contact Angle Data of Nanocellulose Film Depending on Amount of Surface Modifier

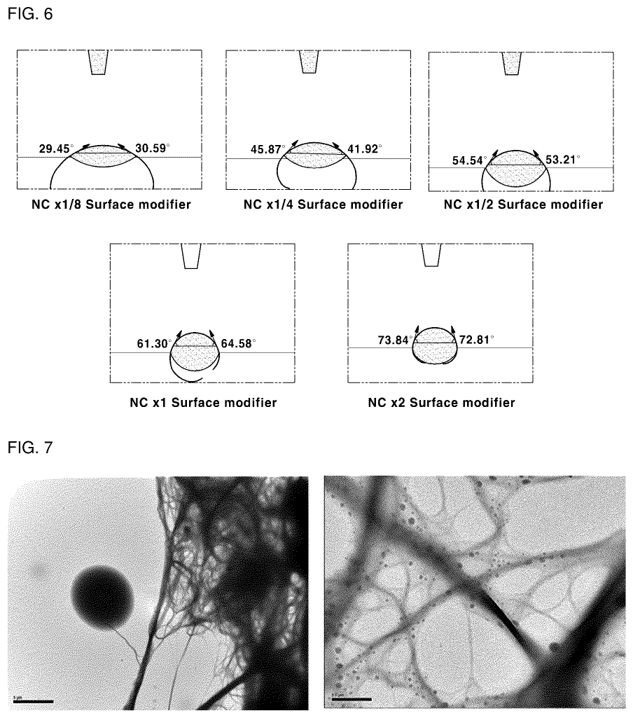

(1) In Preparation Example 1 and FIG. 4, the S.A 4 sample was used.
(2) The material obtained by adding the S.A 4 sample in each of amounts corresponding to ⅛, ¼, ½, 1, and 2 times of the weight of the nanocellulose was used. Each film was manufactured, and the contact angle thereof was measured.
(3) In the test method, the contact angle was measured in order to determine the hydrophobic properties of the nanocellulose film depending on a change in the amount of the surface modifier.
(4) The test results were as shown in FIG. 6. Based on the test results, the higher the amount of the surface modifier, the higher the hydrophobicity, and it was possible to attain nanocellulose having various polarities by controlling the amount thereof. Because the cellulose film is able to absorb water over time, it was judged that the accuracy was lowered with time. Based on the results of measurement at the same time period, there was a change with or without the addition of the surface modifier, but no tendency depending on changes in the type or amount of the hydrophilic group was observed.

### Test Example 4. Measurement of a Transmission Electron Microscope (TEM) Image of Polyol Having Surface-Modified Nanocellulose Dispersed Therein

(1) PTMG in which cellulose with a surface modifier (50 wt % based on the amount of cellulose) was dispersed according to the procedure of Preparation Example 2. PTMG in which cellulose without a surface modifier was dispersed as a comparative example were used.
(2) In the test method, cellulose with or without the surface modifier was dispersed in PTMG1000 as a polyol. Then 1 mg of cellulose-dispersed PTMG was sampled with 1 ml of ethanol and distilled water (2:1), dispersed, and placed on a TEM grid, followed by observation with a TEM.
(3) The test results were as shown in FIG. 7. The left side of FIG. 7 showed an image without the nonionic surfactant, in which the polyol was present separately from CNF, but the right side showed that the polyol was uniformly present around CNF. When NC without the surface modifier on the left side was added to polyol, it was not dispersed but agglomerated. When NC made by adding the surface modifier on the right side was added to polyol, it was present in a dispersed form.

### Test Example 5. Evaluation of Results of Dispersion in Polyol

(1) In the test method, the surface modifier was synthesized depending on a change in the hydrophilic group thereof based on a change in the molar ratio of the initiator and glycidol, 50 wt % of the surface modifier based on the amount of cellulose was added and dispersed, and dispersion stability was measured at a precipitation rate under harsh conditions of 80° C. and vacuum pressure.

(In FIG. 8, \textcircled{1} X, \textcircled{2} only Span80, \textcircled{3} Span80:glycidol=1:1, \textcircled{4} Span80:glycidol=1:3, \textcircled{5} Span80:glycidol=1:5)
(2) The test results were as shown in FIG. 8. From the left, the dispersant not added (X), the interaction promoter without a hydroxyl group, the interaction promoter with one hydroxyl group, and the interaction promoters with three and five hydroxyl groups are shown (for each type of polyol, the appropriate number of hydroxyl groups of the interaction promoter and the amount thereof were varied).
(3) Based on the above results, it was confirmed that dispersion stability was the greatest when the surface modifier synthesized using Span80 and glycidol at 1:3 in PTMG1000 was added in an amount of 50 wt % based on the amount of cellulose (\textcircled{4}).

### Test Example 6. Results of Dispersion in Polyol Depending on Amount of Surface Modifier

(1) The test method was the same as in Test Example 5, but the surface modifier synthesized using Span80 and glycidol at 1:3 was added in different amounts based on the weight of cellulose, and was then sufficiently dispersed until water was removed, after which dispersion stability was measured at a precipitation rate under harsh conditions of 80° C. and vacuum pressure.
(2) The test results were as shown in FIG. 9. From the left in FIG. 9, \textcircled{1} 0 wt %, \textcircled{2} 25 wt %, \textcircled{3} 50 wt %, \textcircled{4} 75 wt %, \textcircled{5} 100 wt %, and \textcircled{6} 150 wt % were added based on the amount of cellulose.
(3) Based on the above results, it was confirmed that dispersion stability was the greatest when the surface modifier synthesized using Span80 and glycidol at 1:3 in PTMG1000 was added in an amount of 50 wt % based on the amount of cellulose (\textcircled{3}).

### Test Example 7. Measurement of DSC of Thermoplastic Polyurethane Depending on Amount of Surface-Modified Nanocellulose

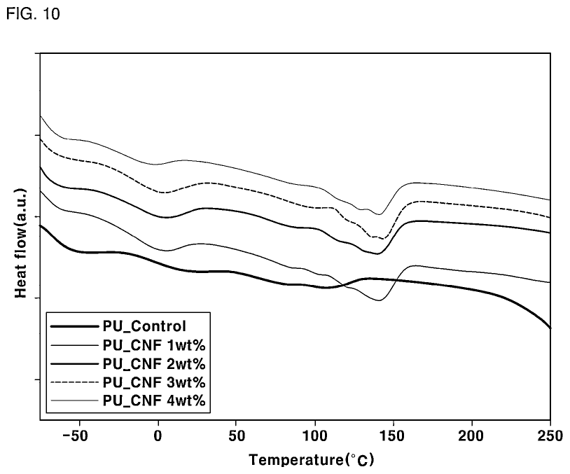

(1) In the test method, 1 wt %, 2 wt %, 3 wt %, and 4 wt % of cellulose added with the surface modifier based on the weight of PTMG, along with a control, were prepared into thermoplastic polyurethane. 2 to 4 mg of the film was added to DSC, the temperature was raised from −70° C. to 240° C. at a rate of 10° C./min, the process was repeated three times, and a second set of data was obtained.
(2) The test results were as shown in FIG. 10.
(3) Based on the above results, it was confirmed that the glass transition temperature corresponding to the amorphous properties of the thermoplastic polyurethane decreased with an increase in the amount of cellulose, which is deemed to be because the added cellulose affects the entanglement of the amorphous properties. Also, the reason why the melting temperature was increased due to the addition of cellulose was that the hydroxyl group of cellulose and the carbonyl group of the urethane group present in the hard segment of polyurethane stably form a hydrogen bond and serve as a composite material.

### Test Example 8. Measurement of TGA and DTA of Thermoplastic Polyurethane Depending on Amount of Surface-Modified Nanocellulose

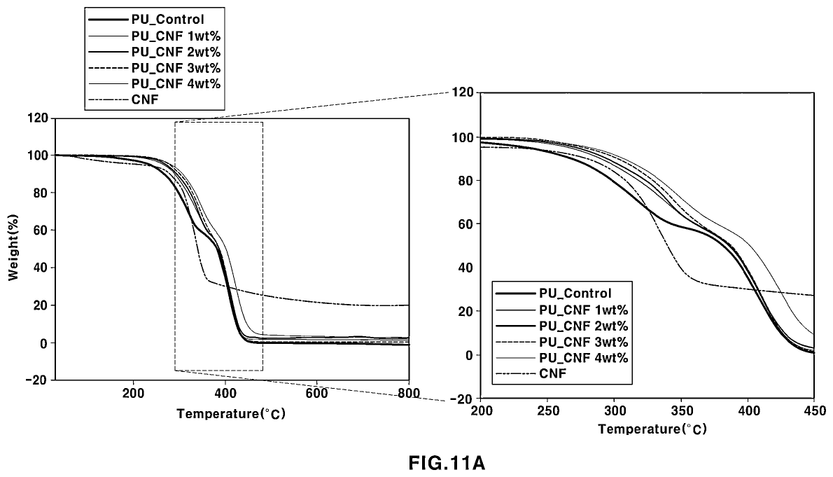

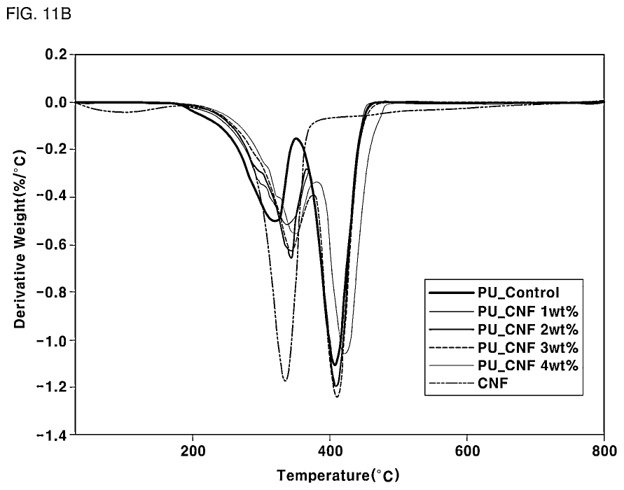

(1) In the test method, 1 wt %, 2 wt %, 3 wt %, and 4 wt % of cellulose added with the surface modifier based on the weight of PTMG, along with a control, were prepared into thermoplastic polyurethane. Also, 7 to 8 mg thereof was placed on a pan and the temperature was raised from 30° C. to 800° C. at a rate of 30° C./min and maintained for 10 minutes. Weight reduction data were obtained.
(2) The test results were as shown in FIGS. 11A and 11B.
(3) Based on the above results, it was confirmed that the two decomposition temperatures were elevated with an increase in the amount of cellulose in the thermoplastic polyurethane film. This is deemed to be because the hydroxyl group of cellulose and the carbonyl group of the urethane group present in the hard segment of polyurethane stably form a hydrogen bond and serve as a composite material, indicating that the decomposition temperature was elevated with an increase in the amount of cellulose.

### Test Example 9. UTM of Thermoplastic Polyurethane Depending on Amount of Surface-Modified Nanocellulose

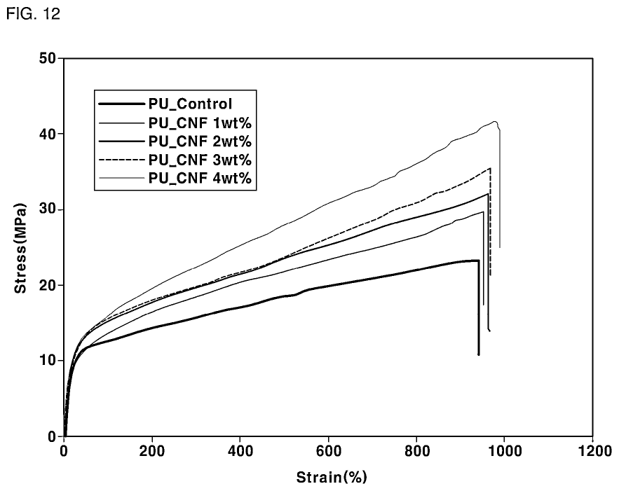

(1) In the test method, each film was manufactured in accordance with a sample-manufacturing method of ASTM standard D638 and was subjected to a tensile strength test using a universal testing machine.
(2) The test results were as shown in FIG. 12.
(3) Based on the results of measurement of the tensile strength of polyurethane (PU) prepared by adding NC using the interaction promoter to polyol, it was confirmed that tensile strength was approximately doubled when 4% NC was added.
(4) Based on the above results, it was confirmed that there was no significant change in tensile strain but the tensile stress was approximately doubled. Thereby, it can be confirmed that the tensile strength is affected by an increase in the amount of cellulose because the hydroxyl group of cellulose and the carbonyl group of the urethane group present in the hard segment of polyurethane stably form a hydrogen bond and serve as a composite material.

As is apparent from the above description, when nanocellulose is prepared using the surface modifier according to one aspect of the present disclosure, precipitation thereof becomes easy in the nanocellulose aqueous solution due to physical hydrogen bonding between the surface modifier and the nanocellulose.

When nanocellulose is prepared using the surface modifier according to one aspect of the present disclosure, dispersibility thereof in the polymer matrix can be increased due to the hydrophobic properties of the alkyl chain of the surface modifier physically bound to the nanocellulose.

When nanocellulose and a polymer matrix including the same are prepared using the surface modifier according to one aspect of the present disclosure, the number of hydroxyl groups in the surface modifier can be adjusted, thus increasing dispersibility and improving physical properties depending on the type of polymer matrix.

When nanocellulose is prepared using the surface modifier according to one aspect of the present disclosure, the surface of nanocellulose is modified only through physical interaction, so side reactions can be minimized in subsequent processes.

When nanocellulose is prepared using the surface modifier according to one aspect of the present disclosure, the surface modification process can be performed without a chemical reaction at a relatively low temperature using an inexpensive starting material. Thereby, a surface modification process for a large amount of nanocellulose can be conducted.

When nanocellulose is prepared using the surface modifier according to one aspect of the present disclosure, a variety of commercially available nonionic surfactants having hydroxyl, carboxyl and amine groups are used as a starting material, so nanocellulose can be appropriately dispersed in various polymers.

When nanocellulose and a polymer matrix including the same are prepared using the surface modifier according to one aspect of the present disclosure, an excellent effect of increasing heat resistance is exhibited.

When nanocellulose and a polymer matrix including the same are prepared using the surface modifier according to one aspect of the present disclosure, an excellent effect of increasing tensile strength is exhibited.

The disclosure has been described in detail with reference to embodiments thereof. However, it should be appreciated by those having ordinary skill in the art that changes may be made in these embodiments without departing from the principles or spirit of the disclosure, the scope of which is defined in the appended claims and their equivalents.

What is claimed is:

1. A method of preparing surface-modified nanocellulose, the method comprising:
mixing a nanocellulose aqueous solution with a nanocellulose surface modifier;
centrifuging a mixed solution; and
removing an amount of water after centrifugation,
wherein the nanocellulose surface modifier is a nonionic surfactant in which a hydrophilic group is subjected to ring opening with glycidol.

2. The method of claim 1, wherein the nonionic surfactant is prepared through a ring-opening reaction of at least one compound selected from among a carboxylic acid compound having 4 to 25 carbon atoms, an alcohol compound having 4 to 25 carbon atoms, and an amine compound having 4 to 25 carbon atoms with glycidol.

3. The method of claim 1, wherein the nonionic surfactant is prepared through a ring-opening reaction of sorbitan monooleate with glycidol.

4. The method of claim 2, wherein the carboxylic acid compound having 4 to 25 carbon atoms is at least one compound selected from among butyric acid, valeric acid, caproic acid, enatic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, alpha-linolenic acid, stearidonic acid, eicosapentaenoic acid, docosahexaenoic acid, linoleic acid, gamma-linoleic acid, dihomo-gamma-linolenic acid, arachidonic acid, docosatetraenoic acid, palmitoleic acid, vaccenic acid, paulinic acid, oleic acid, elaidic acid, gondoic acid, erucic acid, nervonic acid, and mead acid,
wherein the alcohol compound having 4 to 25 carbon atoms is at least one compound selected from among butanol, pentanol, hexanol, heptanol, octanol, nonanol, decanol, undecanol, dodecanol, tridecanol, tetradecanol, pentadecanol, palmityl alcohol, octadecanol, nonadecanol, eicosanol, heneicosanol, docosanol, tricosanol, tetracosanol, and pentacosanol, and
wherein the amine compound having 4 to 25 carbon atoms is at least one compound selected from among butanamine, pentanamine, hexanamine, heptanamine, octanamine, nonanamine, decanamine, undecanamine, dodecanamine, tridecanamine, tetradecanamine, pentadecanamine, hexadecanamine, heptadecanamine, octadecanamine, nonadecanamine, eicosanamine, heneicosanamine, docosanamine, tricosanamine, tetracosanamine, and pentacosanamine.

5. The method of claim 1, wherein the nanocellulose surface modifier is in a hyperbranched polyglycidol (HBPG) form.

6. The method of claim 1, wherein the nanocellulose surface modifier is prepared by reacting at least one compound selected from among a carboxylic acid compound having 4 to 25 carbon atoms, an alcohol compound having 4 to 25 carbon atoms, and an amine compound having 4 to 25 carbon atoms with glycidol at a molar ratio of 1:1-30.

7. The method of claim 5, wherein the nanocellulose surface modifier is prepared by reacting a carboxylic acid compound having 4 to 25 carbon atoms and glycidol at a molar ratio of 1:1-8.

8. The method of claim 1, wherein the nanocellulose surface modifier has 1 to 10 hydroxyl groups.

9. The method of claim 1, wherein nanocellulose in the nanocellulose aqueous solution and the nanocellulose surface modifier are mixed at a weight ratio of 1:0.2-3.
